SEQUENCE LISTING (12 sequences)

SEQ ID NO: 1 - protein, 448 aa, synthetic construct
EVQLVESGGGLVQPGGSLRLSCAASGFSFSTYGMSWVRQAPGKKLEWVATISYGYTYTYYPDSVKGRFTISRDTSKNTLYLQMNSLRAEDTAMYYCVRREDHGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 118 aa, synthetic construct
EVQLVESGGGLVQPGGSLRLSCAASGFSFSTYGMSWVRQAPGKKLEWVATISYGYTYTYYPDSVKGRFTISRDTSKNTLYLQMNSLRAEDTAMYYCVRREDHGAMDYWGQGTLVTVSS

SEQ ID NO: 3 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 5 aa, synthetic construct
TYGMS

SEQ ID NO: 5 - protein, 15 aa, synthetic construct
SYGYTYTYYPDSVKG

SEQ ID NO: 6 - protein, 9 aa, synthetic construct
REDHGAMDY

SEQ ID NO: 7 - protein, 214 aa, synthetic construct
DIQITQSPSSLSASVGDRVTITCRASQNIGTDIQWYQQKPGKSPKLLIKYASESISGVPSRFSGSGSGTDFTLTISSLQPEDFATYYCQQSYSWPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 107 aa, synthetic construct
DIQITQSPSSLSASVGDRVTITCRASQNIGTDIQWYQQKPGKSPKLLIKYASESISGVPSRFSGSGSGTDFTLTISSLQPEDFATYYCQQSYSWPTTFGQGTKVEIK

SEQ ID NO: 9 - protein, 107 aa, synthetic construct [sequence omitted]

SEQ ID NO: 10 - protein, 11 aa, synthetic construct
RASQNIGTDIQ

SEQ ID NO: 11 - protein, 7 aa, synthetic construct
YASESIS

SEQ ID NO: 12 - protein, 9 aa, synthetic construct
QQSYSWPTT

(19) United States
(12) Patent Application Publication
(10) Pub. No.: US 2025/0304660 A1
(43) Pub. Date: Oct. 2, 2025

(54) ANTI-SHIGA MONOCLONAL ANTIBODY AND USES THEREOF

(71) Applicant: The United States of America, as Represented by the Secretary of Agriculture, Washington, DC (US)

(72) Inventors: XIAOHUA HE, RICHMOND, CA (US); ANNA C. HUGHES, SAN DIEGO, CA (US); MARINA E. KIRKLAND MITCHELL, BERKELEY, CA (US)

(21) Appl. No.: 18/640,340

(22) Filed: Apr. 19, 2024

Related U.S. Application Data
(60) Provisional application No. 63/572,588, filed on Apr. 1, 2024.

(51) Int. Cl. C07K 16/12; A61K 39/40; A61K 45/06; A61P 31/04

(52) U.S. Cl. CPC C07K 16/1232; A61K 39/40; A61K 45/06; A61P 31/04; C07K 2317/24

(57) ABSTRACT

The disclosure relates to humanized antibodies (Hu mAb) or antigen-binding fragments thereof that bind to or recognize Shiga toxin (Stx)2, compositions comprising such antibodies or antigen-binding fragments, methods comprising such antibodies or antigen-binding fragments, and methods of using such antibodies or antigen-binding fragments. The Hu mAb or antigen binding fragments thereof that bind to or recognize Stx2 may be used for treating a subject in need thereof to prevent or ameliorate one or more symptoms of Shiga toxin-producing Escherichia coli (STEC) infection in the subject, or to prevent or ameliorate one or more symptoms of hemolytic uremic syndrome (HUS) in the subject.

### CROSS-REFERENCE TO RELATED APPLICATIONS

This application claims priority to U.S. Provisional Patent Application 63/572,588, filed Apr. 1, 2024. The contents of this provisional application is expressly incorporated herein by reference in its entirety.

### FIELD OF THE INVENTION

The disclosure relates to humanized antibodies (Hu mAb) or antigen-binding fragments thereof that bind to or recognize Shiga toxin (Stx)2, compositions comprising such antibodies or fragments thereof, and methods of using such antibodies or fragments thereof.

### SEQUENCE LISTING

The instant application contains a Sequence Listing XML required by 37 C.F.R. § 1.831(a) which has been submitted in XML file format via the USPTO patent electronic filing system, and is hereby incorporated by reference in its entirety. The XML file was created on Mar. 27, 2024, is named Sequence_Listing-003923, and has 13,000 bytes.

### BACKGROUND OF THE INVENTION

Shiga toxin-producing Escherichia coli (STEC) is a major risk to food safety in agricultural sectors of dairy, meat production, and leafy greens. Shiga toxin (Stx), the predominant cause of STEC-associated hemolytic uremia syndrome (HUS), is a potent protein transport and ribosome re-entry inhibitor. In the state of California, there were 9,489 reported STEC infections resulting in 398 cases of HUS from 2013 through 2019. Children 12 and under are disproportionately at risk of developing HUS as they currently constitute 70% of STEC-related HUS cases. It is reported that individuals with typical HUS have a 12% risk of mortality or end-stage renal disease (ESRD) yet 25% of those who 23 recover have long term renal sequelae.

STEC possess a number of virulence factors, but Shiga toxins (Stxs) were considered the most critical in disease pathogenesis and are responsible for HC and HUS. Stxs are comprised of one A subunit (32 kDa) and five B subunits (7.7 kDa). The Stx A subunit is an enzymatically active N-glycosidase that inhibits the activity of rRNA by cleavage of an adenine base from the 28S rRNA component of the eukaryotic ribosomal 60S subunit, causing protein synthesis to cease resulting in cell death. The Stx B subunit is responsible for binding to host cells through interaction with globotriaosylceramide (Gb3) or globotetraosylceramide (Gb4) receptors present on cell surfaces, leading to subsequent internalization of the toxin. There are two serologically distinct groups of Stxs, Stx1 and Stx2. Epidemiological and molecular typing studies suggested that STEC strains expressing Stx2 were more virulent than strains expressing either Stx1 or both Stx1 and Stx2. In contrast to Stx1, many variants of Stx2 have been identified, and these variants differ from each other in terms of their affinity for host receptors, cytotoxicity, and pathogenicity.

Currently, therapeutic interventions against STEC infection are limited since antibiotic treatment may induce overexpression of Stx. Human and humanized antibodies against Stx2 have been described, but they have not shown to be effective in clinical trials.

WO 2010/115278, published Oct. 10, 2010, discloses compositions and methods for stimulating an immune response against Shiga toxin-producing E. coli antigens. The compositions include a fusion protein comprising more than one epitope of an immunogenic STEC protein from more than one STEC serotype. Additional compositions include at least two purified STEC proteins, wherein the STEC proteins are selected from a full-length STEC protein, an immunogenic fragment or variant thereof, wherein at least one of the STEC proteins generates antibodies that react with STEC 0157 and at least one other STEC serotype.

U.S. Pat. No. 5,955,293 relates to antigenic peptides or proteins related to Shiga toxin (ST), Shiga-like toxin I (SLT-I), Shiga-like toxin II (SLT-II) or SLT-II variants. I, and to a vaccine formulation containing such a peptide or protein useful in treating a disease associated with the toxin.

U.S. Pat. No. 7,910,096 discloses human and humanized monoclonal antibodies that specifically bind to Shiga-like toxin II subunit A. The antibodies are said to be neutralizing antibodies against hemolytic uremic syndrome, and useful for treating gnotobiotic piglets infected with E. coli 0157: H7.

U.S. Pat. No. 8,293,245 discloses Stx1 polypeptides that include the 13C4 monoclonal antibody epitope. This patent also discloses methods of treating a subject having, or at risk of developing, a Shiga toxin-associated disease. U.S. Pat. Nos. 8,969,529; 9,801,931 disclose Stx2 polypeptides that include the 11E10 monoclonal antibody epitope. These patents also disclose methods of treating a subject having, or at risk of developing, a Shiga toxin-associated disease.

U.S. Pat. Nos. 9,310,368 and 9,513,287 disclose mouse monoclonal antibodies for the detection of Shiga toxin 2 (Stx2).

Thus, new humanized antibodies useful in controlling STEC infection are needed.

### SUMMARY OF THE INVENTION

The disclosure relates to humanized monoclonal antibodies (Hu mAb) or antigen-binding fragments thereof that bind to or recognize Shiga toxin (Stx) 2, compositions comprising such antibodies, and methods of using them.

In an embodiment, the disclosure relates to an antibody or antigen binding fragment thereof, wherein the antibody or antigen binding fragment thereof comprises a heavy chain variable domain (VH) comprising a complementary determining region VH CDR1 of the amino acid sequence set forth in SEQ ID NO: 4, a VH CDR2 of the amino acid sequence set forth in SEQ ID NO: 5, and a VH CDR3 of the amino acid sequence set forth in SEQ ID NO: 6; and a light chain variable domain (VL) comprising a complementary determining region VL CDR1 of the amino acid sequence set forth in SEQ ID NO: 9, a VL CDR2 of the amino acid sequence set forth in SEQ ID NO: 10, and a VL CDR3 of the amino acid sequence set forth in SEQ ID NO: 11.

In some embodiments of the disclosure, the VH domain comprises the amino acid sequence of SEQ ID NO: 2. In some embodiments of the disclosure, the VL domain comprises the amino acid sequence of SEQ ID NO: 8. In some embodiments, the antibody or antigen-binding fragment is a is humanized monoclonal antibody. In some embodiments of the disclosure, the antibody or antigen-binding fragment thereof binds to a Shiga toxin (Stx) 2 protein.

In an embodiment, the disclosure relates to a host cell expressing an antibody or antigen-binding fragment thereof that binds to an Stx2 protein. In some embodiments, the disclosure relates to a method of producing an antibody or antigen-binding fragment thereof that binds to an Stx2 protein. The method comprising culturing the host cell expressing an antibody or antigen-binding fragment thereof that binds to an Stx2 protein under conditions wherein the antibody or antigen fragment thereof that binds to Stx2 is expressed, and harvesting the antibody or antigen binding fragment thereof.

In an embodiment, the disclosure relates to a pharmaceutical composition comprising an antibody or antigen fragment thereof that binds to Stx2 and a pharmaceutically acceptable carrier. In some embodiments of the disclosure, the pharmaceutical composition comprising an antibody or antigen fragment thereof that binds to Stx2 and a pharmaceutically acceptable carrier further comprises an antibody.

In an embodiment, the disclosure relates to a method of treating a subject to prevent or ameliorate one or more symptoms of Shiga toxin-producing Escherichia coli (STEC) infection in a subject in need thereof, the method comprising administering to the subject an effective amount of a pharmaceutical composition comprising an antibody or antigen binding fragment thereof that binds to an Stx2 protein and a pharmaceutically-acceptable carrier. In some embodiments of the invention, the one or more symptoms of STEC infection treated is one or more symptoms of hemolytic uremic syndrome (HUS).

In an embodiment, the disclosure relates to a kit for diagnostic, prognostic, or therapeutic use, the kit comprising an antibody or antigen binding fragment thereof that binds to an Stx2 protein.

### BRIEF DESCRIPTION OF THE DRAWINGS

FIG. 1A to FIG. 1C depict graphs of the detection and neutralization of Stx2a in vitro. FIG. 1A shows the chemiluminescence in relative luminescent units (RLU) resulting after Vero cells were co-cultured with PBS, Stx1a, or Stx2a, and/or Hu-mAb 2-5 as an indication of cytotoxicity. FIG. 1B shows the ELISA results for detecting 1 ng of wt Stx2a with Hu-mAb 2-5 or mAb 2-5 in relative luminescence units. FIG. 1C shows the percent change in detection of different quantities of wt Stx2a using Hu mAB 2-5 (humanized) or mAb 2-5 (mouse). N=3. The Y axis shows the luminescence in RRLU, and the X Axis shows the different treatments.

FIG. 2A to FIG. 2C depict graphs of ex vivo human PBMC-based CRS and antigen activation assays with Hu-mAb 2-5. FIG. 2A shows a graph of the whole blood CRS assays where whole blood was activated with an adjuvant cocktail of 100 ng/ml of LPS and 10 μM of R848 then co-incubated with 1 μg of antibody, and the TNF-α concentration of supernatant collected 6 and 18 hours post-inoculation was quantified using ELISA. N=7. The Y axis shows the TNF-α in picograms per mL (pg/mL). Statistical analyses used was the Kruskal-Wallis test.

FIG. 2B shows a graph of the T-cell dependent production of IFN-γ+ cells in the population of CD3+CD8+ cells after stimulation. The Y axis shows the percent of IFN-γ+ cells. Statistical analyses used were unpaired Student's t-test. FIG. 2C shows a graph of IFN-γ+ or TNF-α+ in the population after treatment. The Y axis show the percent IFN-γ+ or TNF-α+. For FIG. 2B and FIG. 2C, the percentages plotted were normalized to MOG to remove non-specific activation. N=4-5 . . . . Representative of three independent experiments. Statistical analyses used were unpaired Student's t-test. In FIG. 2A to FIG. 2C the X axis shows the treatments used in the assays. P>0.05 (ns); P≤0.01 (**).

FIG. 3A to 3C depict graphs of the in vivo protection of the murine model from hemolytic uremic syndrome when using Hu-mAb 2-5 during Stx2a challenge. FIG. 3A shows the probability of survival for mice challenged with 18 ng of WT Stx2a (3 LD₅₀) and treated with Hu-mAb 2-5 or PBS. N=20. The Y Axis shows the percent probability of survival. Statistics used were the Mantel-Cox test. FIG. 3B shows the percent change in initial weight and day 3 wt of mice challenged with 18 ng of WT Stx2a and treated with Hu-mAb 2-5 or PBS. N=20. FIG. 3C shows the change in weight over time of mice challenged with 18 ng of WT Stx2a and treated with Hu-mAb 2-5 or PBS. N=20. The Y Axis shows the percent (%) change in weight. Statistics used were Students' unpaired t-test. The X Axis shows the treatments used 0.2 μg, 1 μg, 2 μg, 5 μg, of Hu-mAb 2-5 or PBS. P>0.05 (ns); P≤0.01 (**), P≤0.001 (***); P≤0.0001 (****).

FIG. 4 depicts a graph of the blood urea nitrogen (BUN) levels quantified using serum as an indication of kidney damage mitigation in mice challenged with WT Stx2a (1 LD₅₀) and treated with Hu-mAb 2-5. The Y Axis shos the measured BUN in mg/dL. The X Axis shows the treatments and time post-treatment. Statistics used were the Mantel-Cox test. P>0.05 (ns); P≤0.01 (**), P≤0.001 (***); P≤0.0001 (****).

FIG. 5 depicts images of kidneys from mice challenged with WT Stx2a (1 LD₅₀), either untreated, treated with PBS, or treated with Hu-mAb 2-5. The three images on the left are from H&E staining and the three images on the right are from periodic acid-Schiff (PAS) staining. The top two images are from untreated mice, the middle two images are from mice treated with PBS, and the bottom two images are from mice treated with Hu-mAb 2-5.

### BRIEF DESCRIPTION OF THE SEQUENCE LISTING

The nucleotide sequences disclosed in the specification are listed in Table 1, below.

| Sequence Identifier | Type | Description |
|---|---|---|
| SEQ ID NO: 1 | AA | Hu-mAb 2-5 heavy chain |
| SEQ ID NO: 2 | AA | Hu-mAb 2-5 heavy chain variable domain |
| SEQ ID NO: 3 | AA | Hu-mAb 2-5 heavy chain conserved domain |
| SEQ ID NO: 4 | AA | Hu-mAb 2-5 heavy chain CDR1 |
| SEQ ID NO: 5 | AA | Hu-mAb 2-5 heavy chain CDR2 |
| SEQ ID NO: 6 | AA | Hu-mAb 2-5 heavy chain CDR3 |
| SEQ ID NO: 7 | AA | Hu-mAb 2-5 light chain |
| SEQ ID NO: 8 | AA | Hu-mAb 2-5 light chain variable domain |
| SEQ ID NO: 9 | AA | Hu-mAb 2-5 light chain conserved domain |
| SEQ ID NO: 10 | AA | Hu-mAb 2-5 light chain CDR1 |
| SEQ ID NO: 11 | AA | Hu-mAb 2-5 light chain CDR2 |
| SEQ ID NO: 12 | AA | Hu-mAb 2-5 light chain CDR3 |

### DETAILED DESCRIPTION

The disclosure relates to humanized antibodies (Hu mAb) or antigen-binding fragments thereof that bind to or recognize Shiga toxin (Stx) 2, compositions comprising such antibodies or antigen-binding fragments, methods of using such antibodies or antigen-binding fragments and compositions, and kits comprising such antibodies or antigen-binding fragments thereof.

Disclosed herein is a potentially therapeutic humanized mouse monoclonal antibody (Hu-mAb 2-5) targeting Stx2a, the most common Shiga toxin subtype identified from outbreaks. The disclosure demonstrates that Hu-mAb 2-5 has low immunogenicity in healthy adults ex vivo and high neutralizing efficacy in vivo. The Hu-mAb 2-5 protected mice from mortality and STEC-associated hemolytic uremia syndrome (HUS)-related tissue damage similar to the protection provided by the mouse mAB 2-5.

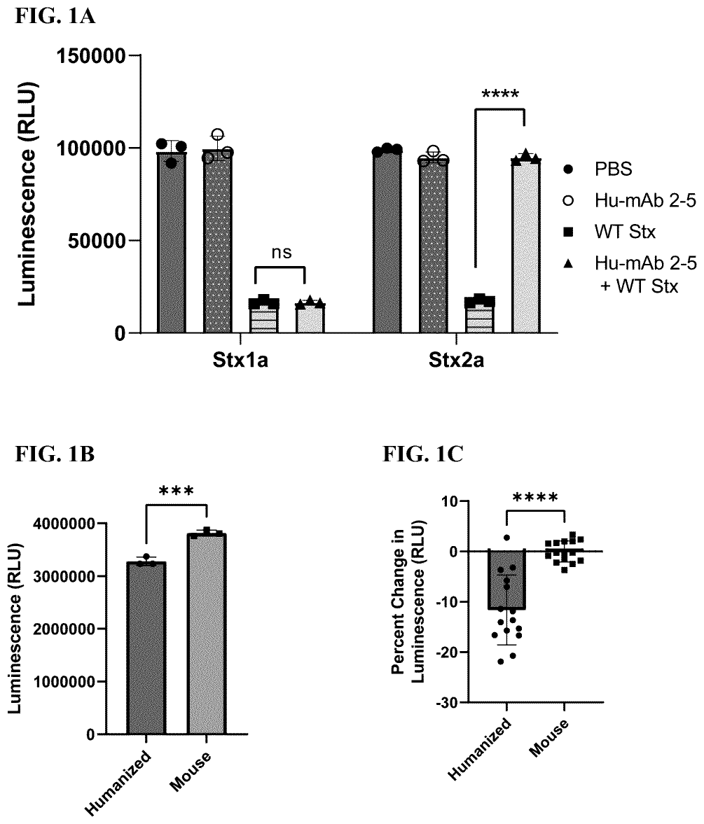

To develop Hu-mAb 2-5, mouse mAb 2-5 was humanized by Creative Biolabs recombinantly in an effort of reducing immunogenicity when administered to human patients. After humanization, there can be a loss of antibody epitope specificity due to replacement of the original mouse framework to human framework in the antibody binding domain. In order to measure loss in efficacy, the Hu mAb 2-5's binding capabilities to Stx2a were quantified using an ELISA at various concentrations of toxin from 1 μg to 100 pg, and the measured luminescence directly compared to the one produced by mAb 2-5. As seen in FIG. 1B and FIG. 1C, the Hu-mAb 2-5 had an approximately 12% loss in efficacy post-humanization. To verify the specificity and potency of Hu-mAb 2-5, Vero cells originating from African green monkey kidneys were co-cultured with either PBS, 10 ng/mL of Stx1a, 10 ng/ml of Stx2a, and/or 20 μg/mL of Hu-mAb 2-5 for one hour at 4° C. before media was replaced with sterile DMEM. After 24 hours at 37° C., cellular cytotoxicity was analyzed using Promega's Cell Titer-Glo® which measures luminescence of ATP released in cell death. From the results shown in FIG. 1A, it was determined that Hu-mAb 2-5 can effectively neutralize Stx2a in vitro and not Stx1a. These results demonstrate that Hu-mAB 2-5 retained specificity for Stx2a.

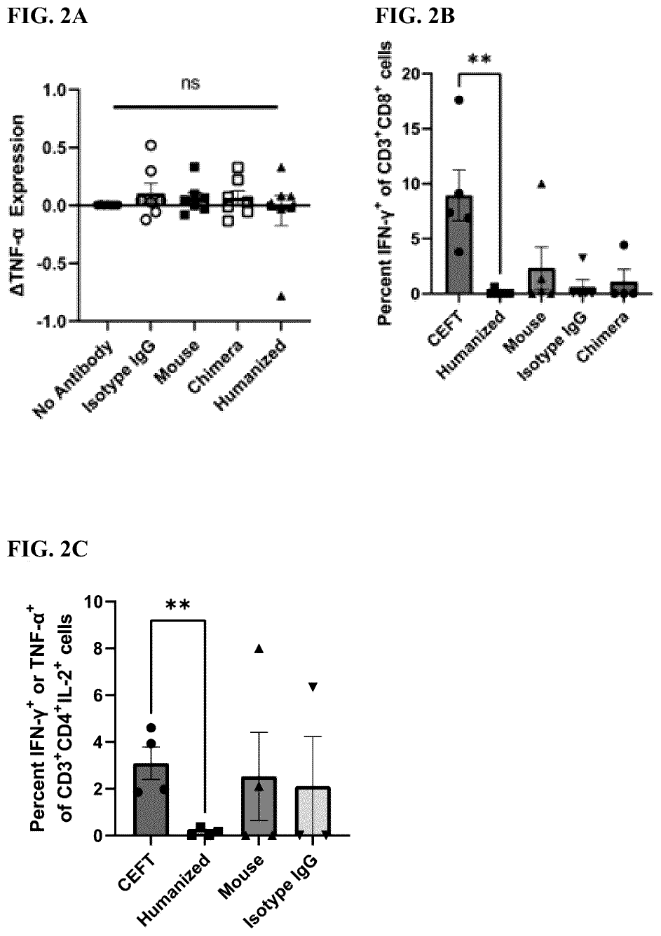

Biologics' therapeutic potential must be counterbalanced with their potential for adverse host reactions such as production of anti-drug antibodies (ADAs), which reduce pharmacogenic efficacy, or induction of cytokine reactive syndrome (CRS), such as that witnessed during 2006's clinical trial for TGN1412, an anti-CD28 m. Thus, Hu-mAb 2-5 was screened for indications of potential reactivity in ex vivo samples from 7 healthy adult human volunteers (4 female and 3 male, ages 27 to 36). Whole blood was co-incubated with an adjuvant cocktail (100 ng/ml of LPS and 10 μM of R848) and 1 μg Hu-mAb 2-5, an Isotype human IgG, mAb 2-5, and chimeric mAb 2-5 (Chi-mAb 2-5, the chimeric intermediate of Hu-mAb 2-5 and mAb 2-5) for 6 hours. Secreted TNF-α were quantified using an ELISA with or without adjuvant. As seen in FIG. 2A, in both conditions Hu-mAb 2-5 did not significantly induce expression of TNF-α above background. Not wishing to be bound by theory, these results suggest a low probability of inducing early hypersensitivity reactions through complement-dependent mechanisms and an absence of antibody-specific effector T-cells from volunteers' immunological memory repertoire. The potential for anti-drug responses from repeated exposure of Hu-mAb 2-5 to ex vivo PBMCs was then examined using a T-cell dependent antigen specific immunogenicity assay originally described by C. Bozkus et al (2021, “A T-cell-based immunogenicity protocol for evaluating human antigen-specific responses,” STAR Protoc. 2:100758). In brief, human PBMCs were treated with GM-CSF, Flt3-L, and IL-4 136 to promote antigen presenting cell (APC) differentiation before co-incubation with media, 1 μM pathogenic peptides CEFT, Hu-mAb 2-5, mAb 2-5, Chi-mAb 2-5, or isotype and adjuvants LPS and R848. Expansion and proliferation of activated naïve T-cells was supported by cytokines IL-2, IL-7, and IL-15 until the 9th day where PBMCs were re-stimulated by either CEFT, corresponding antibodies, human myelin oligodendrocyte glycoprotein (MOG, MHC Class I specific antigen), or potent immunogenic NF-κB activator phorbol myristate acetate (PMA). Re-stimulation with either MOG or PMA after primary stimulation with antibody would identify non-specific activation (MOG) and maximum stimulation potential (PMA). As seen in FIG. 2B and FIG. 2C, little activation of IFN-γ in CD3+CD8+ cells; TNF-α and/or IFN-γ in CD3+CD4+IL2+ was detected after restimulation by Hu-mAb 2-5. Oppositely, repeated stimulation by CEFT had upwards of 9 times greater activation. Interestingly, the immunogenicity profile of mAb 2-5 was relatively low despite its murine origins.

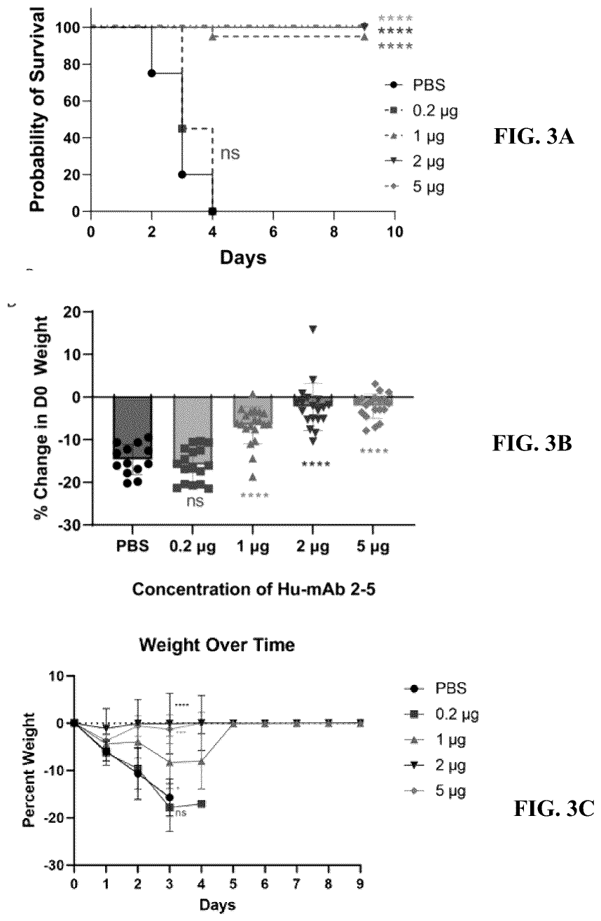

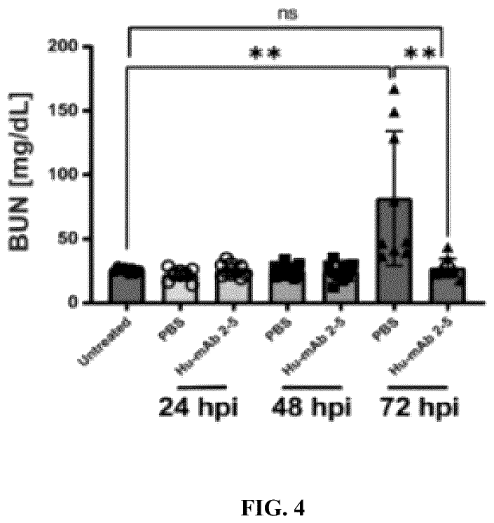

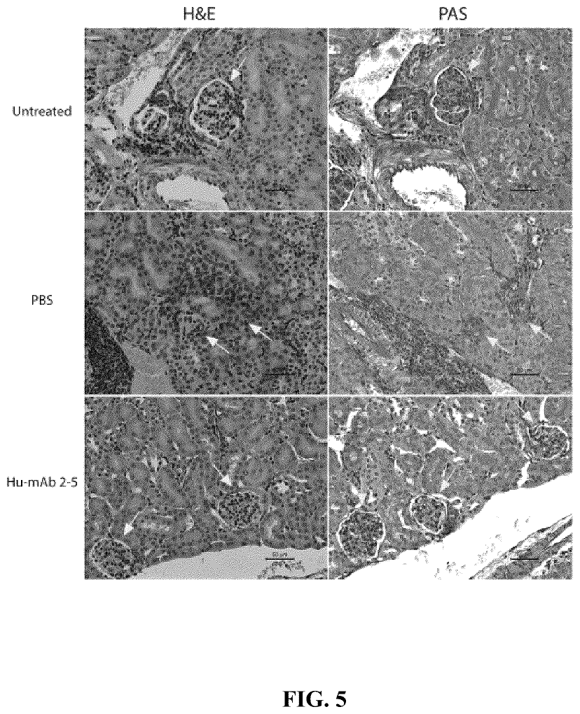

To demonstrate that humanization of mAb 2-5 did not impair its ability to neutralize Stx2a in vivo, Swiss Webster (CFW) mice were inoculated with 18 ng of Stx2a in PBS (3×LD50 i.p.) by i.v. injection 30 minutes after treatment (i.v.) with either PBS, 0.2, 1, 2, or 5 μg of Hu-mAb 2-5, as previously described. As shown in FIG. 3A, treatment with Hu-mAb 2-5 imparted a 90% chance of survival at 1 μg as opposed to 100% of 1 μg mAb 2-5 in CFW mice as previously disclosed (Skinner C. et al., 2015, “New Stx2e monoclonal antibodies for immunological detection and distinction of Stx2 subtypes,” PLOS One 10: e0132419; Cheng L. et al., 2013, “Mouse in vivo neutralization of Escherichia coli Shiga toxin 2 with monoclonal antibodies,” Toxins 5:1845-1858). FIG. 3B shows that dosages of 2 μg and 5 μg were completely protective against mortality by Stx2a and 5 μg resulted in the least changes in weight. To examine Hu-mAb 2-5's ability to mitigate Stx2a induced-kidney damage, mice were inoculated with 6 ng of Stx2a (1 LD50 i.p.) or PBS by i.v. 30 minutes after injection (i.v.) with either 1 μg of Hu-mAb 2-5 or PBS. Mice were euthanized at 24, 48, and 72 hours post inoculation (hpi), blood urea nitrogen levels quantified (BUN), a biomarker for kidney damage, and kidneys were analyzed via histology. As seen in FIG. 4, by 72 hpi, BUN levels quadrupled in mice who did not receive Hu-mAb 2-5 yet remained stable for mice receiving Hu-mAb 2-5. This is evident in histology images shown in FIG. 5, where focal necrotizingglomerulonephritis in mice exposed to Stx2a is clearly seen. Although PBS-treated mice exhibited severe disruption of glomerular structure and high infiltration of leukocytes. Altogether, the results provided here show that Hu-mAb 2-5 can protect and mitigate long term kidney damage caused by Stx-induced HUS.

The work described herein serves as a major stepping stone toward a safe and efficacious monoclonal antibody-based therapy to neutralize Stx2a, potentially in combination with antibiotic therapies during STEC infections preventing the onset of HUS. As seen in FIG. 3A, treatment with Hu-mAb 2-5 resulted in complete protection of mice given a lethal dosage of toxin with a low dosage of antibody (2 μg). The images on FIG. 6 show that one microgram of antibody was sufficient to prevent the destruction of glomeruli in the renal cortex when mice were subjected to one LD₅₀ of Stx2a. This treatment allowed for the maintenance of normal kidney function. In addition to this, Hu-mAb 2-5 demonstrated low immunogenicity immediately after human PBMC exposure and after repeated exposures (as seen in FIG. 2A to FIG. 2C). This proof-of-concept study provides strong evidence for a comprehensive preclinical evaluation for clinical trials and establishes a pipeline for preclinical screening of therapeutic candidates.

There are several Stx-targeting therapeutic antibodies published to date, but none have been approved by the FDA for use in humans (M. Bitzan, et al., 2009, “Safety and Pharmacokinetics of Chimeric Anti-Shiga Toxin 1 and Anti-Shiga Toxin 2 Monoclonal Antibodies in Healthy Volunteers,” Antimicrob. Agents Chemother. 53 (7): 3081-3087). Three have been tested in clinical trials: In Germany in 1999 a polyclonal antibody that recognizes Stx1 and Stx2 was tested (E. L. López et al., 2010, “Safety and Pharmacokinetics of Urtoxazumab, a Humanized Monoclonal Antibody, against Shiga-Like Toxin 2 in Healthy Adults and in Pediatric Patients Infected with Shiga-Like Toxin-Producing Escherichia coli,” Antimicrob. Agents Chemother. 54 (1): 239-243), a chimeric antibody that recognizes Stx1b and Stx2a was tested in 2009 in the U.S. and Canada (P. Doshi, 2014, “From promises to policies: is big pharma delivering on transparency?” BMJ 348: g1615; doi: 10.1136/bmj.g1615)), and in 2010 a humanized antibody that recognizes Stx2b was tested in Argentina (E. Mayo-Wilson et al., 2015, “Are manufacturers sharing data as promised?” BMJ 351: h4169; doi: 10.1136/bmj.h4169).

). All three reported good prognosis with treated groups and mild to relatively no adverse effects which imparts a lack of clarity as to why development has not been finalized (59-61). It has been noted that a lack of qualifying patients for Phase III trials may be an issue (55). Eculizumab, a monoclonal C5 inhibitor, is a therapeutic antibody approved for use during atypical HUS which involves dysregulation of complement activation although its use in STEC-induced HUS is not beneficial and is expensive. The nature of STEC-induced HUS would require a patient to undergo a one-time infusion of neutralizing antibody, likely in combination with antibiotics.

In summary, a humanized monoclonal antibody was developed that can target and effectively neutralize Stx2a in vitro and in vivo. Preliminary immunogenicity screens suggest a low potential for adverse reactions after two exposures. More importantly, its efficacy at neutralizing Stx2a can protect and mitigate tissue damage to kidneys preventing the onset of HUS. In conjunction with antibiotic treatment, Hu-mAb 2-5 may prove to be a critical component of STEC treatment.

Unless otherwise indicated, all numbers expressing quantities of ingredients, properties such as molecular weight, reaction conditions, and so forth as used herein are to be understood as being modified in all instances by the term “about.” Accordingly, unless otherwise indicated, the numerical properties set forth herein are approximations that may vary depending on the desired properties sought to be obtained. Notwithstanding that the numerical ranges and parameters setting forth the broad Scope of the invention are approximations, the numerical values set forth in the specific examples are reported as precisely as possible. Any numerical values, however, inherently contain certain errors necessarily resulting from error found in their respective measurement.

The terms “antibody” and “monoclonal antibody” are used interchangeably herein, and are meant to include intact molecules as well as fragments thereof (such as, for example, Fab and F(ab′)₂ fragments) which are capable of binding. The language “monoclonal antibody” is art-recognized terminology. The humanized monoclonal antibodies of the present invention can be prepared using classical cloning techniques. produce native or human antibodies.

The antibodies disclosed herein can be administered alone or with adjuvants known to one of skill in the art including, but not limited to oil based adjuvants, synthetic adjuvants, and aluminum salts. An oil adjuvant may be, for example, Freunds adjuvant, an aluminum adjuvant may be, for example, MF59, AS01, AS03, AS04, CpG ODN 1018.

The antibodies or fragments thereof disclosed herein that bind to Shiga toxin Stx2 protein may be administered as a single dose, or may be administered in more than one single dose.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

As used herein, the term “effective amount” is meant the amount of antibody or fragment thereof (e.g., a composition comprising antibody or antigen-binding fragment thereof that binds to a Shiga toxin Stx2 protein, optionally containing an antibiotic) required to treat or prevent an infection or disease associated with a Shiga toxin-producing E. coli infection in an individual. The effective amount of drug used to practice the methods described herein for therapeutic or prophylactic treatment of conditions caused by or contributed to by a Shiga toxin-producing E. coli infection varies depending upon the manner of administration, the age, body weight, and general health of the individual. Ultimately, the attending practitioner will decide the appropriate amount and dosage regimen. Such amount is referred to as an “effective amount.”

As used herein, the term “pharmaceutically acceptable” refers to those compounds, materials, compositions, and/or dosage forms, which are suitable for contact with the tissues of an individual (e.g., an animal or a human), without excessive toxicity, irritation, allergic response and other problem complications commensurate with a reasonable benefit/risk ratio.

As used herein, an “antibiotic” is an antimicrobial substance active against bacteria. While STEC should not be treated with antibiotics because antibiotics increase the production of Stxs. The humanized antibody disclosed herein could be tested for use in conjunction with antibiotics during STEC infection since it neutralizes Stxs. This is based on our previous in vitro results (C. Skinner et al. 2015, “An In Vitro Combined Antibiotic-Antibody Treatment Eliminates Toxicity from Shiga Toxin-Producing Escherichia coli,” Antimicrob. Agents Chemother. 59 (9): 5435-5444).

As used herein, the term “pharmaceutical composition” refers to a mixture containing a therapeutic compound to be administered to a subject (e.g., an animal, a human, or a plant), in order to prevent, treat, or control a disease or condition affecting the subject, such as that produced by a Shiga toxin-producing E. coli infection.

As used herein, the term “excipient” refers to a substance formulated alongside the active ingredient of a pharmaceutical composition. At least one excipient may be included, for example, for the purpose of long-term stabilization, or to confer a therapeutic enhancement on the active ingredient in the final dosage form.

As used herein, the term “between” refers to any quantity within the range indicated and enclosing each of the ends of the range indicated.

Other than in the operating examples, or where otherwise indicated, all numbers expressing quantities of ingredients, concentrations, or reaction conditions used herein should be understood as modified in all instances by the term “about.”

As used herein, the term “about” is defined as plus or minus ten percent of a recited value. For example, about 1.0 g means 0.9 g to 1.1 g.

Mention of trade names or commercial products in this disclosure is solely for the purpose of providing specific information and does not imply recommendation or endorsement by the U.S. Department of Agriculture.

While this disclosure may be embodied in many different forms, there are described in detail herein specific preferred embodiments of the invention. The disclosed herein is an exemplification of the principles of the invention and is not intended to limit the invention to the particular embodiments illustrated. All patents, patent applications, scientific papers, and any other referenced materials mentioned herein are incorporated by reference in their entirety. Furthermore, the invention encompasses any possible combination of some or all of the various embodiments and characteristics described herein and/or incorporated herein. In addition, the invention encompasses any possible combination that also specifically excludes any one or some of the various embodiments and characteristics described herein and/or incorporated herein.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Thus, in view of the above, there is described (in part) the following:

An antibody or antigen-binding fragment thereof that binds to or recognizes Stx2, wherein the antibody or antigen-binding fragment thereof comprises a heavy chain variable domain (VH) comprising a complementary determining region VH CDR1 of the amino acid sequence set forth in SEQ ID NO: 4, a VH CDR2 of the amino acid sequence set forth in SEQ ID NO: 5, and a VH CDR3 of the amino acid sequence set forth in SEQ ID NO: 6; and a light chain variable domain (VL) comprising a complementary determining region VL CDR1 of the amino acid sequence set forth in SEQ ID NO: 9, a VL CDR2 of the amino acid sequence set forth in SEQ ID NO: 10, and a VL CDR3 of the amino acid sequence set forth in SEQ ID NO: 11.

The above antibody or antigen-binding fragment thereof, wherein the VH has the amino acid sequence as set forth in SEQ ID NO: 2.

The above humanized antibody or antigen-binding fragment thereof, wherein the VL has the amino acid sequence as set forth in SEQ ID NO: 8.

A host cell expressing the above humanized antibody or antigen-binding fragment thereof.

A method of producing an antibody or antigen-binding fragment thereof that binds to an Stx2 protein, the method comprising culturing the above host cell under conditions wherein the antibody or antigen fragment thereof that binds to Stx2 is expressed, and harvesting the antibody or antigen binding fragment thereof that binds to Stx2.

A pharmaceutical composition comprising the above antibody or antigen fragment thereof and a pharmaceutically-acceptable carrier.

A method of treating a subject in need thereof to prevent or ameliorate one or more symptoms of Shiga toxin-producing Escherichia coli (STEC) infection, the method comprising administering to the subject an effective amount of the above pharmaceutical composition.

A method of treating a subject to prevent or ameliorate one or more symptoms of hemolytic uremic syndrome (HUS), the method comprising administering to the subject an effective amount of the above pharmaceutical composition.

The above method, wherein the antibody or antigen-binding fragment thereof is effective to prevent Stx2a-induced kidney damage.

A pharmaceutical composition comprising the above antibody or antigen fragment thereof, a pharmaceutically-acceptable carrier, and further comprising an antibiotic.

A method of treating a subject in need thereof to prevent or ameliorate one or more symptoms of STEC infection in the subject, the method comprising administering to the subject an effective amount of the above pharmaceutical composition comprising the above antibody or antigen fragment thereof, a pharmaceutically-acceptable carrier, and further comprising an antibiotic.

A method of treating a subject to prevent or ameliorate one or more symptoms of HUS, the method comprising administering to the subject an effective amount of the pharmaceutical composition comprising the above antibody or antigen fragment thereof, a pharmaceutically-acceptable carrier, and further comprising an antibiotic.

A kit for diagnostic, prognostic, or therapeutic use, the kit comprising an antibody or antigen binding fragment thereof that binds to or recognizes Stx2a as taught above.

A polynucleotide encoding the antibody or antigen-binding fragment thereof disclosed herein.

A host cell comprising the above polynucleotide encoding the antibody or antigen-binding fragment thereof disclosed herein.

A vector expressing the polynucleotide or antigen-binding fragment thereof disclosed herein.

An expression vector comprising a DNA molecule or a gene sequence coding the monoclonal antibody or fragment thereof disclosed herein and an expression regulatory sequence operably linked to the DNA molecule or the gene sequence.

A method of making an antibody or antigen binding fragment thereof that binds to or recognizes Stx2; wherein the method comprises culturing the host cell expressing the above humanized antibody or antigen-binding fragment thereof under conditions that allow production of the antibody or antigen binding fragment thereof.

A method of producing an antibody, the method comprising culturing the host cell expressing the above humanized antibody or antigen-binding fragment thereof under conditions suitable for production of the antibody, and harvesting the antibody.

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

### EXAMPLES

Having now generally described this invention, the same will be better understood by reference to certain specific examples, which are included herein only to further illustrate the invention and are not intended to limit the scope of the invention as defined by the claims.

### Example 1

### Materials and Methods

The materials and methods using to prepare a humanized anti-Shiga toxin antibody using mouse monoclonal antibody mAb 2-5, and tests using Siga toxin 2a protein prepared in vitro and purified are described here.

Hu-mab 2-5 production and purification. Plasmids containing sequences for the Hu-mAb 2-5 heavy and light chains were purified from E. coli DH5a using PureLink™ Expi Endotoxin-Free Mega plasmid purification Kit (ThermoFisher, Waltham, Massachusetts, USA) according to manufacturer's protocol. 293F HEK cells cultured in 293F media at 37° C. and 5% CO₂ were transfected with Hu-mAb 2-5 plasmids using 293fectin™ transfection reagent (ThermoFisher) and OPTI-MEM™ medium (ThermoFisher) according to 293F manufacturer's (ThermoFisher) protocol. Culture supernatant was collected after 6 days, and affinity purified using Pierce™ Protein A IgG Purification Kit according to the manufacturer's protocol (ThermoFisher). Fractions containing Hu-mAb 2-5 were further purified using gel filtration on HiLoad™ Superdex 200 chromatography column (trademark registered to Cytva Bioprocess R&D AB, Sweden).

Shiga toxin 2a production and purification. E. coli 10638 was cultured in LB Broth culture medium at 37° C. to an OD of 1 before being induced by mitomycin C at 100 ng/ml, then cultured overnight at 37° C. Cultures were pelleted at 15K×g for 15 minutes at 4° C., and supernatant was sterilized using a 0.22 μm filter. First, 40% ammonium sulfate was added to solution over the course of 10 minutes, then stirred for 30 minutes. Reaction was centrifuged at 12K×g for 15 minutes at 4° C., pellet was discarded. Second, 60% ammonium sulfate was added to the supernatant collected in similar fashion. After centrifugation, the supernatant was discarded, and pellet was resuspended in phosphate buffered saline solution (PBS) and stirred for 1 hour at room temperature (RT) before being centrifuged at 15K×g at 4° C. for 10 minutes. The pellet was discarded. Ammonium sulfate was removed from the supernatant using Zeba™ Spin Desalting Column 7K MWCO, then affinity purification was prepared using AminoLink™ Plus Immobilization Column (ThermoFisher) conjugated with anti-Stx2 antibodies. Fractions containing Stx2a were further purified using gel filtration on a HiLoad™ Superdex™ 200 high-resolution protein fractionation preparative-scale column (trademark registered to Cytva Bioprocess R&D AB, Sweden).

In vitro neutralization assay. All experiments in this study have been repeated at least twice with a minimum sample number of at least 3. Vero cells were cultured in Dulbecco's Modified Eagle Medium (DMEM) supplemented with 1× GlutaMAX™ culture supplement (ThermoFisher) and 10% fetal bovine serum (FBS) at 37° C. and 5% CO₂ and seeded to 96-well tissue culture plates at 100 μL of 0.5×10⁵ cells/mL and allowed to adhere overnight. DMEM containing either 20 μg/mL of Hu-mAb 2-5, 10 ng/mL of Stx1a or Stx 2a, or Hu-mAb 2-5 and Stx1a or Stx2a at their respective concentrations were incubated for 1 hour at 37° C. before being co-incubated with Vero cells for 1 hour at 4° C. Media was removed and replaced with fresh DMEM, cells were incubated for 24 hours at 37° C. and cytotoxicity was assessed using Cell Titer-Glo® Cell Viability Assay (Promega; Madison, Wisconsin, USA) Reagent 1:5 in PBS.

Hu-mAb 2-5 ELISA for Stx2a. One hundred microliters of either Hu-mAb 2-5 or mAb 2-5 was used to coat wells at a concentration of 1 μg/mL in PBS and incubated overnight at 4° C. Plates were washed with 1×TBS with 0.05% Tween 20 polyoxyethylene sorbitol ester (TBST) twice, then blocked with 300 μL of 5% Milk TBST for 1 hour at RT with shaking. Stx1a and Stx2a were diluted in PBS at the following concentrations: 1 μg/mL, 100 ng/ml, 10 ng/ml, 1 ng/67 mL, and 100 μg/mL and added to each well. After incubation for 1 hour at 37° C., plates were washed 6 times in TBST before 100 μL of rabbit anti-Stx2a polyclonal antibody (200 ng/mL) in blocking buffer was added to each well and incubated for 1 hour at RT with shaking. Plates were washed 6 times in TBST. One hundred microliters of goat anti-rabbit HRP (1:10000; Promega) in blocking buffer was added to each well and incubated for 1 hour at RT. Plates were washed 6 times in TBST and 100 μL of SuperSignal Pico chemiluminescence substrate (Thermofisher) was added to each well and luminescence was recorded.

Whole blood cytokine release screen. Whole blood was diluted 1:8 in R10 (RPMI 1640 without glutamine, add 10 vol % fetal calf serum, 2 mM 1-glutamine, 100 IU/mL Penicillin/100 μg/mL Streptomycin) and 100 μL was seeded into a 96-well plate for approximately 1.25×10⁴ PBMCs. One hundred microliters of R10 media with or without adjuvant (LPS [200 ng/ml] and R848 [20 μM]; InvivoGen; San Diego, California, USA) and co-incubated with 2 μM of Human IgG Isotype Control (Invitrogen), Chi-mAb 2-5, Hu-mAb 2-5, or Mo-mAb 2-5 (otherwise referred to as Stx 2-5, mouse mAb 2-5, or Mo-mAb 2-5) for 6 hours at 37° C. and 5% CO₂. The supernatant was collected and an ELISA for TNF-α (Biolegend; San Diego, California, USA) was done according to manufacturer's protocol.

T-cell dependent immunogenicity screening. Human PBMCs were purified from whole blood and seeded at 1×10⁵ cells/mL in X-VIVO 15 cell culture media. One hundred μL of solution containing GM-CSF [2000 IU/mL], IL-4 (1000 IU), and Flt3-L [100 ng/mL] was added and incubated for 24 hours in cell culture conditions. One hundred μL of media was removed and replaced with 100 μL of solution containing adjuvant (R848 [20 μM], LPS [200 ng/ml], and IL-1B [20 ng/mL]) and 1 μM CEFT or antibody. Cells were maintained with R10 media for 5 days supplemented with IL-2 [20 μM], IL-7 [200 ng/mL], and IL-15 [20 ng/ml]. On day 7, R10 media without cytokines was used. On day 9, using R10 media, cells were pooled and re-seeded to 2×10⁵ cells and allowed to rest overnight. Cells were restimulated with R10 media with αCD28 [2 μg/mL] and αCD49d [2 μg/mL] (BD Biosciences) and 1 μM of respective antibodies, CEFT, MOG, and PMA-Ionomycin [50 ng/ml-1 μg/mL]. For intracellular staining, protein transport inhibitors GolgiStop (Monensin) [7.33 μL/mL] and GolgiPlug (Brefeldin A) [11 μL/mL] (BD Biosciences) were added and incubated for 8 hours in cell culture conditions. 90 Cells were then stained extracellularly for CD3-FITC, CD4-BV785, and CD8-APC and viability using ThermoFisher LIVE/DEAD Fixable AQUA. Cells were then fixed and permeabilized for intracellular staining for TNF-α-PE-Cy7, IFN-γ-PE, and IL-2-BV605 and analyzed on BD FACS Aria Fusion cytometer and FlowJo v10.8. Normal whole peripheral blood (PB) was obtained from healthy donors using Institutional Review Board (IRB)-approved consent forms and protocols through the supplier, STEMCELL Technologies.

In vivo Stx2a survival challenge. All animal work was reviewed and approved by the Institution Animal Care and Use committee. Sex-matched Swiss-Webster outbred mice 6-9 weeks old were challenged with 18 ng of WT Stx2a (3 LD₅₀) intravenously 30 minutes after intravenous injection with PBS or 0.2 μg, 1 μg, 2 μg, or 5 μg of Hu-mAb 2-5 under general anesthesia (isofluorane). Weight, temperature, and health condition were monitored for endpoint criteria over the course of 9 days.

In vivo Hu-mAb 2-5 neutralization challenge. Female Swiss-Webster outbred mice 6-7 weeks old were challenged with 6 ng of WT Stx2a (1 LD₅₀) intravenously 30 minutes after intravenous injection of PBS or 1 μg of Hu-mAb 2-5 under general anesthesia (isofluorane). Weight, temperature, and health condition was monitored over the course of the experiment. Mice were euthanized at 24, 48, and 72 hours post-inoculation. Blood was collected via the posterior vena cava, kidneys were collected and preserved in formalin, embedded in paraffin and stained for H&E and periodic acid-Schiff. Serum was used to analyze blood urea nitrogen (BUN) levels using blood urea nitrogen colorimetric assay (Invitrogen).

### Example 2

### Preparation and Testing of Anti-Shiga Toxin Antibody

The mouse monoclonal antibody mAb 2-5 was humanized using a CDR-graft method to obtain Hu-mAb 2-5. The Hu mAb 2-5 was tested for its specificity and efficacy in detecting Stx2.

To reduce immunogenicity when administered to humans, mouse mAb 2-5 was humanized by Creative Biolabs (Shirley, New York, USA). Briefly, humanization was performed by using a CDR-graft method to obtain the TOP 1 humanized antibody candidate After that, expression vectors for both, humanized candidate and mouse/human chimeric IgG were constructed with the variable region of the parent murine antibody, and transient expression was conducted.

The amino acid sequence of the humanized mAb 2-5 (Hu-mAb 2-5) heavy chain is EVOLVESGGGLVQPGGSLRLSCAASGFSFSTYGMSWVRQAPGKKLEWVATISYGYTYT YYPDSVKGRFTISRDTSKNTLYLQMNSLRAEDTAMYYCVRREDHGAMDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLP PSREEMKNQVSLTCLVKGFYPSDIAVEWESNGQPENNYKTTPPVLDSDGSFFLYSKLTV DKSRWQQGNVFSCSVMHEALHNHYTQKSLSLSPGK, and is set forth in SEQ ID NO: 1.

Amino acids 1 to 118 correspond to the heavy chain variable region, which are set forth in SEQ ID NO: 2. Amino acids 119 to 447 correspond to the heavy chain constant region, which are set forth in SEQ ID NO: 3. The heavy chain CDR1 amino acid sequence is TYGMS, and is set forth in SEQ ID NO: 4. The heavy chain CDR2 amino acid sequence is SYGYTYTYYPDSVKG, and is set forth in SEQ ID NO: 5. The heavy chain CDR3 amino acid sequence is REDHGAMDY, and is set forth in SEQ ID NO: 6.

The amino acid sequence of the Hu-mAb 2-5 light chain is DIQITQSPSS LSASVGDRV TITCRASQNIGTDIQWYQQKPGKSPKLLIKYASESISGVPSRFSGSGSGTDFTLTISSLQPE DFATYYCQQSYSWPTTFGQGTKVEIKRTVAAPSVFIFPPSDEQLKSGTASVVCLLNNFYP REAKVQWKVDNALQSGNSQESVTEQDSKDSTYSLSSTLTLSKADYEKHKVYACEVTHQ GLSSPVTKSFNRGEC, and is set forth in SEQ ID NO: 7. Amino acids 1 to 107 correspond to the light chain variable region, which are set forth in SEQ ID NO: 8. Amino acids 108 to 214 correspond to the light chain constant region, which are set forth in SEQ ID NO: 9. The light chain CDR1 amino acid sequence is RASQNIGTDIQ, and is set forth in SEQ ID NO: 10. The light chain CDR2 amino acid sequence is YASESIS, and is set forth in SEQ ID NO: 11. The light chain CDR3 amino acid sequence is QQSYSWPTT, and is set forth in SEQ ID NO: 12.

After humanization, there can be a loss of antibody epitope specificity due to the replacement of the original mouse framework with the human framework in the antibody binding domain (R.-M. Lu, et al., 2020, “Development of therapeutic antibodies for the treatment of diseases,” J. Biomed. Sci. 27:1; C. Marks, et al., 2021, “Humanization of antibodies using a machine learning approach on large-scale repertoire data,” Bioinformatics 37:4041-4047; Y. Safdari et al., 2013 “Antibody humanization methods—a review and update,” Biotechnol. Genet. Eng. Rev. 29:175-186; 2019, “Immunogenicity Assessment for Therapeutic Protein Products 1,” in Immune Aspects of Biopharmaceuticals and Nanomedicines, eds. Bawa, R., Szebeni, J., Webster, T. J. & Audette, G. F. 537-583, Jenny Stanford Publishing). To verify the specificity and potency of Hu-mAb 2-5, Vero cells originating from African green monkey kidneys were co-cultured with either PBS, 10 ng/ml of Stx1a or Stx2a, and/or 20 μg/mL of Hu-mAb 2-5 for 1 hour at 4° C. before media was replaced with sterile Dulbecco's Modified Eagle Medium (DMEM). After 24 hours at 37° C., cellular cytotoxicity was analyzed using Promega's Cell Titer-Glo which measures the luminescence of ATP released in cell death.

To measure loss in efficacy, the Hu-mAb 2-5's binding capabilities to Stx2a were quantified using ELISA at various concentrations of toxin from 1 μg to 100 μg and directly compared luminescence to mAb Stx2-5. It was found that Hu-mAb 2-5 had an approximately 12% loss in efficacy post humanization. The ability of Hu-mab 2-5 to detect and neutralize Stx2a in vitro was assayed. Briefly, Vero cells were co-cultured with either PBS, 10 ng/ml of Stx1a, 10 ng/ml of Stx2a, and/or 20 μg/mL of Hu-mAb 2-5 in media for 1 hour. Media was replaced and cells were allowed to recover for 24 hours before cytotoxicity was assessed using chemiluminescence. As can be seen in FIG. 1A, Hu-mAb 2-5 detected and neutralized Stx2a in vitro, but not Stx1a demonstrating that the humanized antibody retained specificity for Stx2a. The results of an ELISA detecting 1 ng of WT Stx2a in relative luminescence units u (RLU) using Hu-mAb 2-5 or mAb 2-5 are shown in FIG. 1B. N=3. The percent change in detection of different quantities of WT Stx2a (1 μg, 100 ng, 10 ng, 1 ng, and 100 pg) of Hu-mAb 2-5 when compared to mAb 2-5 are shown in FIG. 1C. N=15. Statistics shown were derived from Students' unpaired t-test. P>0.05 (ns); P≤0.001 (***); P≤0.0001 (****). N=3.

Biologics' therapeutic potential must be counterbalanced with their potential for adverse host reactions such as the production of anti-drug antibodies, which reduce pharmacogenic efficacy, or infusion related reactions such as induction of cytokine reactive syndrome (CRS), witnessed during 2006's clinical trial for TGN1412 anti-CD28 mAb (H. Attarwala, 2010, “TGN1412: From Discovery to Disaster,” J. Young Pharm. 2:332-336; H. Gao et al., 2019, “TNF-α promotes human antibody-mediated complement-dependent cytotoxicity of porcine endothelial cells through downregulating P38-mediated Occludin expression,” Cell Commun. Signal. 17:75). Hu-mAb 2-5 was screened for indications of potential reactivity in ex vivo samples from seven healthy adult human volunteers (four females and three males, ages 27 to 36, listed in Table 2 below).

TABLE 2
Donor Profiles for ex-vivo Studies

| | | | | Weight | Height | |
|---|---|---|---|---|---|---|
| Donor | Sex | Age | Ethnicity | (kg) | (cm) | Smoker |
| 1 | M | 33 | Asian | 79 | 178 | No |
| 2 | F | 31 | African | 55 | 160 | No |
| | | | American | | | |
| 3 | F | 30 | Hispanic | 96 | 163 | No |
| 4 | M | 37 | African | 93 | 206 | Yes |
| | | | American | | | |
| 5 | F | 51 | Caucasian | 56 | 160 | No |
| 6 | M | 56 | Caucasian | 63 | 173 | Yes |
| 7 | F | 27 | Mixed | 85 | 155 | No |

Whole blood was co-incubated with an adjuvant cocktail (100 ng/mL of LPS and 10 μM of R848) and 1 μg Hu-mAb 2-5, an Isotype human IgG, mAb 2-5, and chimeric mAb 2-5 (Chi-mAb 2-5, the chimeric intermediate of Hu-mAb 2-5 and mAb 2-5) for 6 hours. Secreted TNF-α were quantified using an ELISA with or without adjuvant and in both conditions. As seen in

FIG. 2A, Hu-mAb 2-5 did not significantly induce expression of TNF-α above background. These results suggest a low probability of inducing early hypersensitivity reactions through complement-dependent mechanisms and an absence of antibody-specific effector T-cells from volunteers' immunological memory repertoire. The potential for anti-drug responses from repeated exposure of Hu-mAb 2-5 to ex vivo PBMCs was then examined using a T-cell dependent antigen-specific immunogenicity assay originally described by Bozkus et al. (Supra). In brief, human PBMCs were treated with GM-CSF, Flt3-L, and IL-4 to promote antigen presenting cell (APC) differentiation before co-incubation with media, 1 μM pathogenic peptides CEFT, Hu-mAb 2-5, mAb 2-5, Chi-mAb 2-5, or isotype and adjuvants LPS and R848. Expansion and proliferation of activated naïve T-cells were supported by cytokines IL-2, IL-7, and IL-15 until the ninth day where PBMCs were re-stimulated by either CEFT, corresponding antibodies, human myelin oligodendrocyte glycoprotein (MOG, MHC Class I specific antigen), or potent immunogenic NF-κB activator phorbol myristate acetate (PMA). Restimulation with either MOG or PMA after primary stimulation with antibodies would identify non-specific activation (MOG) and maximum stimulation potential (PMA). As seen in FIG. 2B and FIG. 2C, little activation of IFN-γ was observed in CD3+CD8+ cells nor TNF-α and/or IFN-γ in CD3+CD4+IL2+ after restimulation by Hu-mAb 2-5. Oppositely, repeated stimulation by CEFT had upwards of nine times greater activation. Interestingly, the immunogenicity profile of mAb 2-5 was relatively low despite its murine origins.

Lastly, humanization of mAb 2-5 does not impair its ability to neutralize Stx2a in vivo. Swiss Webster (CFW) mice were inoculated with 18 ng of Stx2a in PBS (3×LD50 i.p.) by i.v. injection 30 minutes after treatment (i.v.) with either PBS, 0.2, 1, 2, or 5 μg of Hu-mAb 2-5, as previously described (L. Cheng, et al, 2013, “Mouse in Vivo Neutralization of Escherichia coli Shiga Toxin 2 with Monoclonal Antibodies,” Toxins 5:1845-1858). As seen in FIG. 3A, it was found that Hu-mAb 2-5 treatment imparted a 90% chance of survival at 1 μg as opposed to 100% of 1 μg mAb 2-5 in CFW mice. FIG. 3B shows that dosages of 2 μg and 5 μg were completely protective against mortality by Stx2a and 5 μg resulted in the least changes in weight. FIG. 3C shows that mice receiving the antibody had little to no symptoms after being dosed with toxin. This data suggests that the antibody is effective in protecting mice from the toxin.

To examine Hu-mAb 2-5's ability to mitigate Stx2a-induced kidney damage, mice were inoculated with 6 ng of Stx2a (1 LD50 i.p.) or PBS by i.v. 30 minutes after injection (i.v.) with either 1 μg of Hu-mAb 2-5 or PBS. Mice were euthanized at 24, 48, and 72 hours post inoculation (hpi), blood urea nitrogen levels (BUN) were quantified as shown in FIG. 4 (BUN is a biomarker for kidney damage), and the kidneys were analyzed via histology. As seen in FIG. 4, by 72 hpi, BUN levels quadrupled in mice who did not receive Hu-mAb 2-5 yet remained stable for mice receiving Hu-mAb 2-5. It should be noted that BUN levels also increase during dehydration, to which dehydration is directly related to murine renal inflammation and a common symptom of HUS. This is evident by histology images showing focal necrotizing glomerulonephritis (R. L. Siegler et al., 1991, “Long-term outcome and prognostic indicators in the hemolytic-uremic syndrome,” J. Pediatr. 118:195-200) in mice exposed to Stx2a. As seen in FIG. 5, PBS treated mice exhibited focal disruption of glomerular structure and high infiltration of leukocytes, although the mild tubular epithelial injury was only present in placebo treated mice 72 hpi with Stx2a. Overall acute tubular necrosis levels were scored 0 (absent) for all groups except placebo mice 72 hpi with Stx2a which were scored 1 (minimal) by an unblinded pathologist. Altogether, this evidence suggests that Hu-mAb 2-5 can protect and mitigate long-term kidney damage caused by Stx-induced HUS.

We claim:

1. An antibody or antigen-binding fragment thereof that binds to or recognizes Stx2, wherein the antibody or antigen-binding fragment thereof comprises a heavy chain variable domain (VH) comprising a complementary determining region VH CDR1 of the amino acid sequence set forth in SEQ ID NO: 4, a VH CDR2 of the amino acid sequence set forth in SEQ ID NO: 5, and a VH CDR3 of the amino acid sequence set forth in SEQ ID NO: 6; and a light chain variable domain (VL) comprising a complementary determining region VL CDR1 of the amino acid sequence set forth in SEQ ID NO: 9, a VL CDR2 of the amino acid sequence set forth in SEQ ID NO: 10, and a VL CDR3 of the amino acid sequence set forth in SEQ ID NO: 11.

2. The antibody or antigen-binding fragment thereof of claim 1, wherein the VH has the amino acid sequence as set forth in SEQ ID NO: 2.

3. The humanized antibody or antigen-binding fragment thereof of claim 1, wherein the VL has the amino acid sequence as set forth in SEQ ID NO: 8.

4. A host cell expressing the antibody or antigen-binding fragment of claim 1.

5. A method of producing an antibody or antigen-binding fragment thereof that binds to an Stx2 protein, the method comprising culturing the host cell of claim 4 under conditions wherein the antibody or antigen fragment thereof that binds to Stx2 is expressed, and harvesting the antibody or antigen binding fragment thereof that binds to Stx2.

6. A pharmaceutical composition comprising an antibody or antigen fragment thereof of claim 1 and a pharmaceutically-acceptable carrier.

7. A method of treating a subject in need thereof to prevent or ameliorate one or more symptoms of Shiga toxin-producing Escherichia coli (STEC) infection in the subject, the method comprising administering to the subject an effective amount of the pharmaceutical composition of claim 6.

8. A method of treating a subject in need thereof to prevent or ameliorate one or more symptoms of hemolytic uremic syndrome (HUS) in the subject, the method comprising administering to the subject an effective amount of the pharmaceutical composition of claim 6.

9. The method of claim 8, wherein the antibody or antigen-binding fragment thereof is effective to prevent Stx2a-induced kidney damage.

10. The pharmaceutical composition of claim 6, further comprising an antibiotic.

11. A method of treating a subject in need thereof to prevent or ameliorate one or more symptoms of STEC infection in the subject, the method comprising administering to the subject an effective amount of the pharmaceutical composition of claim 10.

12. A method of treating a subject in need thereof to prevent or ameliorate one or more symptoms of HUS in the subject, the method comprising administering to the subject an effective amount of the pharmaceutical composition of claim 10.

13. A kit for diagnostic, prognostic, or therapeutic use, the kit comprising an antibody or antigen binding fragment thereof of claim 1.

14. A polynucleotide encoding the antibody or antigen-binding fragment thereof of claim 1.

15. A vector expressing the polynucleotide of claim 14.

16. An expression vector comprising a DNA molecule or a gene sequence coding the monoclonal antibody or fragment thereof of claim 1 and an expression regulatory sequence operably linked to the DNA molecule or the gene sequence.

17. A host cell comprising the polynucleotide of claim 14.

18. A method of producing an antibody, the method comprising culturing the host cell of claim 17 under conditions suitable for production of the antibody, and harvesting the antibody.

19. A method of making an antibody or antigen binding fragment thereof that binds to or recognizes Stx2; wherein the method comprising culturing the host cell of claim 4 under conditions that allow production of the antibody or antigen binding fragment thereof.
